(19) United States
(12) Patent Application Publication
(10) Pub. No.: US 2026/0224568 A1
(43) Pub. Date: Aug. 6, 2026

(54) Obicetrapib for Treatment of Dementias

(71) Applicant: NewAmsterdam Pharma B.V., Naarden (NL)

(72) Inventors: Marc Ditmarsch, Den Haag (NL); Johannes Jacob Pieter Kastelein, Blaricum (NL); Michael Harvey Davidson, Sunny Isles, FL (US)

(21) Appl. No.: 19/635,622

(22) Filed: Mar. 31, 2026

Related U.S. Application Data
(63) Continuation of application No. 19/370,585, filed on Oct. 27, 2025.
Application No. 19/370,585 is a continuation of application No. 19/185,146, filed on Apr. 21, 2025, now abandoned.
Application No. 19/185,146 is a continuation of application No. 18/827,744, filed on Sep. 7, 2024, now abandoned.
Application No. 18/827,744 is a continuation-in-part of application No. 18/613,486, filed on Mar. 22, 2024, now abandoned.
Application No. 18/613,486 is a continuation-in-part of application No. 18/279,385, filed on Mar. 4, 2022, now abandoned.
(60) Provisional application No. 63/157,586, filed on Mar. 5, 2021.

(51) Int. Cl. A61K 31/506; A61K 9/20; A61P 25/28

(52) U.S. Cl. CPC A61K 31/506; A61K 9/20; A61P 25/28

(57) ABSTRACT

Methods are presented for slowing progression of neurodegenerative disease in a subject who has or is at risk of developing a neurodegenerative disease. The methods comprise administering a therapeutically effective amount of obicetrapib or a pharmaceutically acceptable salt thereof to the subject.

### 1. CROSS-REFERENCE TO RELATED APPLICATIONS

This application is a continuation of U.S. application Ser. No. 19/370,585, filed Oct. 27, 2025, which is a continuation of U.S. application Ser. No. 19/185,146, filed Apr. 21, 2025, which is a continuation of U.S. application Ser. No. 18/827,744, filed Sep. 7, 2024, which is a continuation-in-part of U.S. application Ser. No. 18/613,486, filed Mar. 22, 2024, which is a continuation-in-part of U.S. application Ser. No. 18/279,385, filed Aug. 29, 2023, which is the National Stage of International Application No. PCT/IB2022/000098, filed Mar. 4, 2022, which claims the benefit of and priority to U.S. Provisional Application No. 63/157,586 filed Mar. 5, 2021.

### 2. BACKGROUND OF THE INVENTION

Neurodegenerative diseases, and particularly neurodegenerative diseases that result in cognitive impairment and dementia, are a serious burden on patients, their families, and society. Alzheimer's Disease and other dementias have been calculated to have cost 2.55 million disability-adjusted life-years (DALYs) in the United States in 2017. See “Burden of neurological disorders across the US from 1990-2017,” JAMA Neurol. 78(2):165-176 (2021). The burden is expected to increase as the median age of the population increases.

Despite the need for effective treatments, no disease-modifying treatments capable of slowing progression of neurodegenerative diseases, particularly degenerative diseases that result in cognitive impairment and dementia, have been unambiguously demonstrated to be effective. In addition, patients with cognitive impairment present compliance challenges for treatments that must be administered chronically, and/or administered using a patient-controlled device. Treatments that require parenteral administration impose additional impediments. There is, therefore, a need for disease-modifying treatments capable of slowing progression of neurodegenerative diseases, particularly degenerative diseases that result in cognitive impairment and dementias, and that can be administered orally.

### 3. SUMMARY OF THE INVENTION

The ε4 allele of the apolipoprotein E gene (APOE4) has long been known to be a risk factor for late-onset Alzheimer's disease (AD). Although the mechanism by which this genotype increases AD risk remains unclear, the strength of this association positions lipoprotein metabolism as central to the pathology of AD.

Studies of natural allelic variants of cholesteryl ester transfer protein (CETP) have demonstrated an association between CETP function with memory decline and incidence of dementia, Sanders et al., JAMA 303(2):150-158 (2010), and the brain-penetrant CETP inhibitor, evacetrapib, has recently been shown to rescue the memory deficit in mice that are double-transgenic for human amyloid precursor protein (APP) and CETP, Phenix and Munter, AAIC21 Conference Poster. Obicetrapib, a potent inhibitor of cholesterol esterase transfer protein (CETP), has previously been shown to increase HDL levels and ApoE levels in plasma. We now show that obicetrapib crosses the blood-brain barrier, with measurable CNS accumulation following oral administration.

Accordingly, a method is provided for slowing progression of neurodegenerative disease in a subject who has or is at risk of developing a neurodegenerative disease. The method comprises administering a therapeutically effective amount of obicetrapib or a pharmaceutically acceptable salt thereof to the subject.

In some embodiments, obicetrapib or salt thereof is administered orally. In various embodiments, obicetrapib is administered once per day. In certain embodiments, the dose is 5-25 mg po QD of obicetrapib or salt thereof, such as 5 mg po QD, 10 mg po QD, 15 mg po QD, 20 mg po QD, or 25 mg po QD.

In some embodiments, obicetrapib or salt thereof is administered once daily for at least 8 weeks, at least 6 months, at least 12 months, or at least 24 months.

In some embodiments, the subject has been diagnosed with or is at risk for Alzheimer's Disease (AD), Lewy Body dementia, vascular or multi-infarct dementia, or frontotemporal dementia (FTD). In particular, embodiments, the subject has been diagnosed with or is at risk for developing AD. In specific embodiments, the subject has been diagnosed with AD. In certain embodiments, the subject has at least one ApoE4 allele or two ApoE4 alleles. In specific embodiments, the method comprises the prior step of detecting ApoE4 alleles in the subject.

In some embodiments, the subject has been diagnosed with mild cognitive impairment (MCI).

In some embodiments, the subject has been determined, prior to treatment, to have at least one of (i) decreased levels of Aβ42 in plasma, (ii) increased levels of Aβ40 in plasma, (iii) decreased ratio of Aβ42/Aβ40 in plasma, (iv) increased levels of neurofilament light (NfL) in plasma, and (v) increased levels of neurogranin in plasma, as compared to a healthy control population that does not have and that is not at elevated risk for neurodegenerative disease.

In some embodiments, the subject has been determined, prior to treatment, to have at least one of (i) decreased levels of Aβ42 in CSF, (ii) increased levels of A$40 in CSF, (iii) decreased ratio of Aβ42/Aβ40 in CSF, (iv) increased levels of neurofilament light (NFL) in CSF, and (v) increased levels of neurogranin in CSF, as compared to a healthy control population that does not have and that is not at elevated risk for neurodegenerative disease.

In some embodiments, the subject has been determined, prior to treatment, to have abnormal patterns of Tau based on Tau PET imaging.

In some embodiments, the dose of obicetrapib or salt thereof is sufficient to prevent at least one of (i) further decrease in levels of Aβ42 in plasma, (ii) further increase in levels of Aβ40 in plasma, (iii) further decrease in ratio of Aβ42/Aβ40 in plasma, (iv) further increase in levels of neurofilament light (NFL) in plasma, and (v) further increase in levels of neurogranin in plasma, as compared to levels immediately prior to start of treatment.

In some embodiments, the dose of obicetrapib or salt thereof is sufficient to prevent at least one of (i) further decrease in levels of Aβ42 in CSF, (ii) further increase in levels of Aβ40 in CSF, (iii) further decrease in ratio of Aβ42/Aβ40 in CSF, (iv) further increase in levels of neurofilament light (NFL) in CSF, and (v) further increase in levels of neurogranin in CSF, as compared to levels immediately prior to start of treatment.

In typical embodiments, obicetrapib is administered as a tablet. In certain embodiments, the tablet comprises obicetrapib as the calcium salt.

### 4. DETAILED DESCRIPTION OF THE INVENTION

### 4.1. Definitions

Unless defined otherwise, all technical and scientific terms used herein have the meaning commonly understood by a person skilled in the art to which this invention belongs.

The terms “subject” or “individual” are used interchangeably and refer to an animal to be treated, including but not limited to humans and non-human primates; rodents, including rats and mice; bovines; equines; ovines; felines; and canines.

The term “patient” refers to a human subject.

The terms “treating”, “treatment”, and grammatical variations thereof are used in the broadest sense understood in the clinical arts. Accordingly, the terms do not require cure or complete remission of disease, and encompass obtaining any clinically desired pharmacologic and/or physiologic effect.

The phrase “therapeutically effective amount” refers to the amount of a compound that, when administered to a subject for treating a disease, condition, or disorder, is sufficient to effect treatment of the disease, condition, or disorder.

### 4.2. Other Interpretational Conventions

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises”, “comprising”, “containing”, “having”, “includes”, “including” and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art. Unless otherwise specified, “about” intends ±10% of the stated value. Where a percentage is provided with respect to an amount of a component or material in a composition, the percentage should be understood to be a percentage based on weight, unless otherwise stated or understood from the context.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of R-enantiomer and S-enantiomer, and enantio-enriched stereomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

### 4.3. Methods of Treatment

In a first aspect, methods are provided for slowing progression of neurodegenerative disease in a subject who has or is at risk of developing a neurodegenerative disease. The methods comprise administering a therapeutically effective amount of obicetrapib or a pharmaceutically acceptable salt thereof to the subject.

### 4.3.1. Obicetrapib

Obicetrapib, formerly known as TA-8995, is a compound of formula (I):

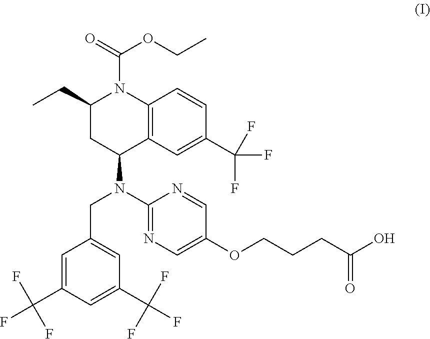 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

(2R,4S){[3,5Bis(trifluoromethyl)benzyl]-[5(3-carboxypropoxy)pyrimidin-2-yl]amino}-2-ethyl-6-trifluoromethyl-3,4-dihydro-2H-quinoline-1-carboxylic acid ethyl ester
   Methods of synthesizing obicetrapib are known. See, e.g., U.S. Pat. Nos. 7,872,126; 8,084,611; and 10,112,904, the disclosures of which are incorporated herein by reference in their entireties.

### 4.3.2. Neurodegenerative Diseases

In some embodiments, the subject has been diagnosed with or is at risk for Alzheimer's Disease (AD), Lewy Body dementia, vascular or multi-infarct dementia, or frontotemporal dementia (FTD).

In certain embodiments, the subject has been diagnosed with or is at risk for developing AD.

In certain embodiments, the subject has been diagnosed with AD. In specific embodiments, the subject has been diagnosed with AD based on the NIA-AA Research Framework criteria, with biomarker classification of A⁺T⁺N⁺ or A⁺T⁺N⁻ based upon (i) CSF profile consistent with AD (an Aβ42 concentration of <1000 μg/mL AND p-tau>19 μg/mL, and/or (ii) a ratio of p-tau/Aβ42 of ≥0.024). In specific embodiments, the biomarkers are measured by Elecsys assay.

In certain embodiments, the subject has been diagnosed with AD based on documented amyloid positron emission tomography (PET) scan evidence. In certain embodiments, the subject has AD Clinical Stage 3 or 4. In specific embodiments, the subject has AD clinical stage 3 or 4 based on the NIA-AA Research Framework criteria: (i) a mini-mental state examination (MMSE) score>20 inclusive, and/or Clinical Dementia Rating scale-Sum of Boxes (CDR-SB) global score≥0.5 and ≤1 with memory box score≥1.0.

In certain embodiments, the subject is at risk for developing AD. In particular embodiments, the subject has at least one ApoE4 allele. In particular embodiments, the subject has two ApoE4 alleles. In some embodiments, the method further comprises the prior step of detecting the presence of ApoE4 alleles in the subject.

In some embodiments, the subject has been diagnosed with mild cognitive impairment (MCI). In certain embodiments, the diagnosis of MCI is made using the Short Test of Mental Status, the Montreal Cognitive Assessment (MoCA) or the Mini-Mental State Examination (MMSE).

In various embodiments, the subject has been determined prior to treatment to have at least one of (i) decreased levels of Aβ42 in plasma, (ii) increased levels of Aβ40 in plasma, (iii) decreased ratio of Aβ42/Aβ40 in plasma, (iv) increased levels of neurofilament light (NFL) in plasma, and (v) increased levels of neurogranin in plasma, as compared to a healthy control population that does not have neurodegenerative disease.

In various embodiments, the subject has been determined prior to treatment to have at least one of (i) decreased levels of Aβ42 in CSF, (ii) increased levels of Aβ40 in CSF, (iii) decreased ratio of Aβ42/Aβ40 in CSF, (iv) increased levels of neurofilament light (NFL) in CSF, and (v) increased levels of neurogranin in CSF, as compared to a healthy control population that does not have and that is not at elevated risk for neurodegenerative disease.

In various embodiments, the subject has been determined prior to treatment to have abnormal patterns of Tau based on Tau PET imaging.

### 4.3.3. Dose Regimen

In various embodiments, obicetrapib or salt thereof is administered orally. In typical embodiments, obicetrapib is administered as a tablet for oral administration. In various embodiments, the dose of obicetrapib or salt thereof is 2.5-25 mg by mouth per day (2.5-25 mg po QD). In some embodiments, the dose of obicetrapib or salt thereof is 5-20 mg by mouth per day (5-20 mg po QD). In some embodiments, the dose of obicetrapib or salt thereof is 10-20 mg by mouth per day (10-20 mg po QD). In specific embodiments, the daily dose is 2.5 mg po QD, 5 mg po QD, 10 mg po QD, 15 mg po QD, 20 mg po QD or 25 mg po QD. In various embodiments, the dose is administered once per day. In some embodiments, the dose is divided and the 2.5-25 mg total daily dose, or 5-20 mg total daily dose, or 10-20 mg total daily dose, is administered as a plurality of divided doses.

In various embodiments, obicetrapib is administered as a tablet. In some embodiments, the tablet comprises 2.5 mg, 5 mg, 7.5 mg, 10 mg, 15 mg, 20 mg or 25 mg of obicetrapib or pharmaceutically acceptable salt thereof. In some embodiments, the tablet contains obicetrapib as the calcium salt. In particular embodiments, the table contains 5 mg obicetrapib as a calcium salt.

In specific embodiments, tablets are round, 6 mm in diameter, white film-coated tablets, containing 5 mg of obicetrapib as the calcium salt. In specific embodiments, tablets are round, 6 mm in diameter, white film-coated tablets, containing 10 mg of obicetrapib as the calcium salt. In specific embodiments, the excipients present in the tablet cores are microcrystalline cellulose, mannitol, sodium starch glycollate, colloidal silicon dioxide, and magnesium stearate. In specific embodiments, a commercially available film-coating formula (Opadry II white, ex Colorcon) is applied to the cores.

In various embodiments, obicetrapib or salt thereof is administered once daily for at least 8 weeks, at least 6 months, at least 12 months, at least 24 months, or at least 36 months.

### 4.4. Further Embodiments

### 4.4.1. Dose Regimen

In various embodiments, obicetrapib or pharmaceutically acceptable salt thereof is administered in an amount effective to increase levels of total ApoE-HDL in blood as compared to the level prior to commencement of treatment. In typical embodiments, blood levels of ApoE-HDL are measured in plasma. In preferred embodiments, obicetrapib or salt thereof is administered in an amount effective to increase plasma levels of ApoE-HDL in particles lacking ApoC3 and/or percentage of plasma HDL-ApoE in HDL particles lacking ApoC3.

### 4.4.2. Clinical Endpoints

In various embodiments, the dose of obicetrapib or salt thereof is sufficient to prevent at least one of (i) further decrease in levels of Aβ42 in plasma, (ii) further increase in levels of Aβ40 in plasma, (iii) further decrease in ratio of Aβ42/Aβ40 in plasma, (iv) further increase in levels of neurofilament light (NFL) in plasma, and (v) further increase in levels of neurogranin in plasma, as compared to levels prior to start of treatment.

In various embodiments, the dose of obicetrapib or salt thereof is sufficient to prevent at least one of (i) further decrease in levels of Aβ42 in CSF, (ii) further increase in levels of Aβ40 in CSF, (iii) further decrease in ratio of Aβ42/Aβ40 in CSF, (iv) further increase in levels of neurofilament light (NFL) in CSF, and (v) further increase in levels of neurogranin in CSF, as compared to levels immediately prior to start of treatment.

### 4.4.3. Further Numbered Embodiments

1. A method of slowing progression of neurodegenerative disease in a subject who has or is at risk of developing a neurodegenerative disease, comprising:
administering a therapeutically effective amount of obicetrapib or a pharmaceutically acceptable salt thereof to the subject.
2. The method of embodiment 1, wherein the amount is effective to increase levels of total ApoE HDL in blood.
3. The method of embodiment 1 or embodiment 2, wherein the amount is effective to increase levels of ApoE-HDL in blood.
4. The method of embodiment 3, wherein the amount is effective to increase levels of ApoE-HDL in particles lacking ApoC3 in blood.
5. The method of any one of embodiments 1-4, wherein obicetrapib or salt thereof is administered orally.
6. The method of embodiment 5, wherein obicetrapib is administered once per day.
7. The method of embodiment 6, wherein the dose is 5-20 mg po QD of obicetrapib or salt thereof.
8. The method of embodiment 7, wherein the dose is 5 mg po QD.
9. The method of embodiment 7, wherein the dose is 10 mg po QD.
10. The method of embodiment 7, wherein the dose is 15 mg po QD.
11. The method of embodiment 7, wherein the dose is 20 mg po QD.
12. The method of any one of embodiments 7-11, wherein obicetrapib or salt thereof is administered once daily for at least 8 weeks.
13. The method of embodiment 12, wherein obicetrapib or salt thereof is administered once daily for at least 6 months.
14. The method of embodiment 13, wherein obicetrapib or salt thereof is administered once daily for at least 12 months.
15. The method of embodiment 14, wherein obicetrapib or salt thereof is administered once daily for at least 24 months.
16. The method of any one of embodiments 1-15, wherein the subject has been diagnosed with or is at risk for Alzheimer's Disease (AD), Lewy Body dementia, vascular or multi-infarct dementia, or frontotemporal dementia (FTD).
17. The method of embodiment 16, wherein the subject has been diagnosed with or is at risk for developing AD.
18. The method of embodiment 17, wherein the subject has been diagnosed with AD.
19. The method of embodiment 17 or embodiment 18, wherein the subject has at least one ApoE4 allele.
20. The method of embodiment 19, wherein the subject has two ApoE4 alleles.
21. The method of any one of embodiments 19-20, further comprising the prior step of detecting ApoE4 alleles in the subject.
22. The method of any one of embodiments 1-21, wherein the subject has been diagnosed with mild cognitive impairment (MCI).
23. The method of any one of embodiments 1-22, wherein the subject has been determined prior to treatment to have at least one of (i) decreased levels of Aβ42 in plasma, (ii) increased levels of Aβ40 in plasma, (iii) decreased ratio of Aβ42/Aβ40 in plasma, (iv) increased levels of neurofilament light (NFL) in plasma, and (v) increased levels of neurogranin in plasma, as compared to a healthy control population that does not have and that is not at elevated risk for neurodegenerative disease.
24. The method of any one of embodiments 1-23, wherein the subject has been determined prior to treatment to have at least one of (i) decreased levels of Aβ42 in CSF, (ii) increased levels of Aβ40 in CSF, (iii) decreased ratio of Aβ42/Aβ40 in CSF, (iv) increased levels of neurofilament light (NFL) in CSF, and (v) increased levels of neurogranin in CSF, as compared to a healthy control population that does not have and that is not at elevated risk for neurode generative disease.
25. The method of any one of embodiments 1-24, wherein the subject has been determined prior to treatment to have abnormal patterns of Tau based on Tau PET imaging.
26. The method of any one of embodiments 1-25, wherein the dose of obicetrapib or salt thereof is sufficient to prevent at least one of (i) further decrease in levels of Aβ42 in plasma, (ii) further increase in levels of Aβ40 in plasma, (iii) further decrease in ratio of Aβ42/Aβ40 in plasma, (iv) further increase in levels of neurofilament light (NFL) in plasma, and (v) further increase in levels of neurogranin in plasma, as compared to levels immediately prior to start of treatment.
27. The method of any one of embodiments 1-26, wherein the dose of obicetrapib or salt thereof is sufficient to prevent at least one of (i) further decrease in levels of Aβ42 in CSF, (ii) further increase in levels of Aβ40 in CSF, (iii) further decrease in ratio of Aβ42/Aβ40 in CSF, (iv) further increase in levels of neurofilament light (NFL) in CSF, and (v) further increase in levels of neurogranin in CSF, as compared to levels immediately prior to start of treatment.
28. The method of any one of embodiments 1-27, wherein obicetrapib is administered as a tablet.
29. The method of embodiment 28, wherein the tablet comprises obicetrapib as the calcium salt.
30. The method of embodiment 28 or 29, wherein the tablet comprises one or more excipients.
31. The tablet of embodiment 30 wherein the one or more excipients comprises microcrystalline cellulose, mannitol, sodium starch glycollate, colloidal silicon dioxide, and magnesium stearate or combinations thereof.

### 5. EXAMPLES

### 5.1. Example 1: Qualitative Whole Body Analysis Demonstrates that Obicetrapib Crosses the Blood-Brain Barrier

To determine the tissue distribution of obicetrapib and/or its metabolites post-dose, and particularly to determine distribution into brain tissues, we employed the use of radiolabeled [¹⁴C]-obicetrapib and quantitative whole-body techniques.

The mice were maintained in rooms at a temperature of 19 to 25° C., with a relative humidity of between 40 and 70%, and exposed to fluorescent light (nominal 12 hours) each day. They were permitted free access to a Western-type diet (semi-synthetic modified diet containing 15% cacao butter, 1% corn oil and 40.5 sucrose). Equal numbers of male (n=5) and female mice (n=5) were given a single administration by gavage at 5 g/kg of [¹⁴C]-obicetrapib. The mice were humanely sacrificed at 1 hour, 4 hours, 24 hours, 72 hours and 168 hours from time of dosing, following which blood and tissue samples were retrieved.

Using blood plasma as the reference point, the data (see Tables 1-4 below) indicate that in female mice 1 hour post-dose, the concentration of [¹⁴C]-obicetrapib in tissues of the brain is about 4% the concentration in plasma. At the 4 hour mark, that relative concentration rises significantly to about 16% of the concentration in plasma. Specifically, in the brain choroid plexus, the radiolabeled drug product is found in concentrations almost 50% of blood plasma concentrations. Also notable is an observed relative concentration of almost 17% in the cerebellum. Compared with the female mice, the male counterparts displayed similar overall concentrations in the brain, while showing slightly lower concentration in the cerebellum. These data indicate that at 4 hours after an oral dose of 5 g/kg there is significant obicetrapib distribution into the brain.

TABLE 1
Concentration Following Oral Dose Of [¹⁴C]-Obicetrapib In Female Mice
(Concentration Units ngEq/g)

| | Female | Female | Female | Female | Female |
|---|---|---|---|---|---|
| Sample | 1 h | 4 h | 24 h | 72 h | 168 h |
| Blood | 3890 | 6940 | 7710 | 1790 | BLQ |
| (QWBA) | | | | | |
| Plasma (LSC) | 8520 | 13100 | 13800 | 3250 | 182 |
| Brain | 380 | 1910 | 1040 | BLQ | BLQ |
| Brain | 422 | 2210 | 1090 | BLQ | BLQ |
| cerebellum | | | | | |
| Brain | 456 | 2010 | 1010 | BLQ | BLQ |
| cerebrum | | | | | |
| Brain choroid | 1910 | 5600 | 1590 | BLQ | BLQ |
| plexus | | | | | |
| Brain medulla | 384 | 1780 | 1130 | BLQ | BLQ |
| Brain | 356 | 1980 | 908 | BLQ | BLQ |
| olfactory lobe | | | | | |
| Brain | 406 | 2090 | 996 | BLQ | BLQ |
| thalamus | | | | | |

TABLE 2
Tissue: Plasma Concentration Ratios Following
Oral Dose Of [¹⁴C]-Obicetrapib In Female Mice

| | Female | Female | Female | Female | Female |
|---|---|---|---|---|---|
| Sample | 1 h | 4 h | 24 h | 72 h | 168 h |
| Blood | 0.456 | 0.529 | 0.559 | 0.553 | NC |
| (QWBA) | | | | | |
| Plasma (LSC) | 1.00 | 1.00 | 1.00 | 1.00 | 1.00 |
| Brain | 0.0446 | 0.146 | 0.0754 | NC | NC |
| Brain | 0.0495 | 0.168 | 0.0789 | NC | NC |
| cerebellum | | | | | |
| Brain | 0.0536 | 0.153 | 0.0730 | NC | NC |
| cerebrum | | | | | |
| Brain choroid | 0.224 | 0.427 | 0.116 | NC | NC |
| plexus | | | | | |
| Brain medulla | 0.0451 | 0.136 | 0.0819 | NC | NC |
| Brain | 0.0417 | 0.151 | 0.0659 | NC | NC |
| olfactory lobe | | | | | |
| Brain | 0.0477 | 0.159 | 0.0723 | NC | NC |
| thalamus | | | | | |

TABLE 3
Concentration Following Oral Dose Of [¹⁴C]-Obicetrapib In Mice
(Concentration Units ngEq./g)

| | Male | Male | Male | Male | Male |
|---|---|---|---|---|---|
| Sample | 1 h | 4 h | 24 h | 72 h | 168 h |
| Blood | 3130 | 5710 | 6100 | 2420 | BLQ |
| (QWBA) | | | | | |
| Plasma (LSC) | 5530 | 12700 | 11600 | 4490 | 395 |
| Brain | 331 | 1640 | 1020 | BLQ | BLQ |
| Brain | 381 | 1820 | 1060 | BLQ | BLQ |
| cerebellum | | | | | |
| Brain | 336 | 1720 | 1000 | BLQ | BLQ |
| cerebrum | | | | | |
| Brain choroid | 1380 | 8040 | 2440 | 490 | BLQ |
| plexus | | | | | |
| Brain medulla | 333 | 1620 | 1050 | BLQ | BLQ |
| Brain | BLQ | 1570 | 894 | BLQ | BLQ |
| olfactory lobe | | | | | |
| Brain | 380 | 1650 | 1020 | BLQ | BLQ |
| thalamus | | | | | |

TABLE 4
Tissue: plasma concentration ratios following
oral dose of [¹⁴C]-Obicetrapib in Mouse

| | Male | Male | Male | Male | Male |
|---|---|---|---|---|---|
| Sample | 1 h | 4 h | 24 h | 72 h | 168 h |
| Blood | 0.566 | 0.449 | 0.524 | 0.539 | NC |
| (QWBA) | | | | | |
| Plasma (LSC) | 1.00 | 1.00 | 1.00 | 1.00 | 1.00 |
| Brain | 0.0598 | 0.129 | 0.0877 | NC | NC |
| Brain | 0.0690 | 0.143 | 0.0907 | NC | NC |
| cerebellum | | | | | |
| Brain | 0.0609 | 0.135 | 0.0861 | NC | NC |
| cerebrum | | | | | |
| Brain choroid | 0.249 | 0.632 | 0.209 | 0.109 | NC |
| plexus | | | | | |
| Brain medulla | 0.0602 | 0.127 | 0.0901 | NC | NC |
| Brain | NC | 0.123 | 0.0768 | NC | NC |
| olfactory lobe | | | | | |
| Brain | 0.0687 | 0.130 | 0.0875 | NC | NC |
| thalamus | | | | | |

### 5.2. Example 2: a Phase 2a, Open-Label, Multicenter Study to Confirm the Efficacy and Safety of Obicetrapib in Participants with Early Alzheimer's Disease (Hetero/Homozygote E4 Carriers)

### 5.2.1. Study Design and Duration

This study is a multi-center, proof of concept, Phase 2a study in participants with early AD to confirm the efficacy, safety, and tolerability of obicetrapib therapy. Study duration for individual participants is approximately 24 weeks (excluding Screening and Follow-up).

### 5.2.2. Objectives

One objective of this study is to confirm the effect of obicetrapib in participants with early Alzheimer's Disease (AD) on levels of ApoA-I, ApoE, and cholesterol efflux capacity in cerebrospinal fluid (CSF). Another objective is to confirm the safety and tolerability of obicetrapib in participants with early AD. A further objective is to confirm the following:
To confirm the effect of obicetrapib in participants with early AD on lipoprotein profiles (pharmacodynamics [PD]).
To confirm the effect of obicetrapib in participants with early AD on biomarkers involved with neurodegeneration and inflammation.
To confirm the effect of obicetrapib in participants with early AD on other PD exploratory biomarkers.
To confirm the efficacy of obicetrapib in participants with early AD in delaying disease progression compared to standard of care.
To confirm the efficacy of obicetrapib in participants with early AD on cognition.

### 5.2.3. Inclusion Criteria

Participants who meet all of the following criteria are eligible to participate in the study:
1. Age range: 50-75 years of age at the Screening Visit
2. Males, or females who are post-menopausal or otherwise not of child-bearing potential
3. Diagnosis of AD based on the NIA-AA Research Framework criteria:
   a. Biomarker classification A+T+N+ or A+T+N− based upon:
      i. CSF profile consistent with AD (an Aβ42 concentration of <1000 μg/mL AND p-tau>19 μg/mL, or a ratio of p-tau/Aβ42 of ≥0.024 (Elecsys assay)) taken during the Screening period prior to the day of the first dose of study medication or,
      ii. Documented evidence of a CSF profile consistent with AD obtained within the previous 12 months, or
      iii. Documented amyloid positron emission tomography (PET) scan evidence acquired within the previous 12 months
4. AD Clinical Stage 3 or 4 based on the NIA-AA Research Framework criteria
   a. Have a mini-mental state examination (MMSE) score at screening and baseline>20 inclusive
   b. Clinical Dementia Rating scale-Sum of Boxes (CDR-SB) global score≥0.5 and ≤1 with memory box score≥1.0
5. Able to speak, read and write the local language fluently
6. Have an APOE genotype of E4/E4 or E3/E4
7. Patients should either be:
   a. Not treated with any approved treatments for AD with a reasonable expectation that, based on the course of illness, need for treatment is not imminent and the patient should not be initiated on treatment for the length of the study, or
   b. Stabilized on an approved medication(s) for the treatment of AD for at least 3 months prior to baseline. The dose of the AD treatment should remain the same after entering the study.
8. Patient and care partner are willing to consent to all study procedures

### 5.2.4. Exclusion Criteria

Participants who meet any of the following criteria are excluded from participation in the study:
1. Other than AD, neurologic or medical disorder which may impair cognition including: head trauma, seizure disorder, neurodegenerative disease, hydrocephalus, cerebral/spinal hematoma, inflammatory disease, central nervous system infection (e.g., encephalitis or meningitis), neoplasm, toxic exposure, metabolic disorder (including hypoxic or hypoglycemic episodes), or endocrine disorder, or any significant medical conditions that, in the opinion of the investigator, prohibits their participation in the study.
2. Any contra-indication to undergo magnetic resonance imaging (MRI), as judged by local Principal Investigator (PI) or radiologist.
3. MRI of the brain indicative of significant abnormality, including, but not limited to, prior hemorrhage or infarct>1 cm³, >3 lacunar infarcts, deep white matter lesions corresponding to a Fazekas score of 3, cerebral contusion, encephalomalacia, aneurysm, vascular malformation, subdural hematoma, hydrocephalus, space-occupying lesion (eg, abscess or brain tumor such as meningioma). Small incidental meningiomas may be allowed if discussed and approved by the Medical Monitor.
4. History of any of the following neurological, psychiatric or medical conditions:
   a. History of large vessel stroke
   b. History of myocardial infarction or unstable angina within the previous 12 months
   c. Or other clinical manifestations of atherosclerotic vascular disease
   d. Type 1 diabetes and uncontrolled type 2 diabetes (hemoglobin A1c [HbA1c]>8%)
   e. Systemic blood pressure>150/90 mmHg on 3 separate determinations
   f. History of hyperaldosteronism
   g. Significant renal or hepatic dysfunction
   h. Current or previous hepatitis B infection (defined as positive test for hepatitis B surface antigen (HbSAg) and/or hepatitis B core antibody (anti-HBc). Subjects with immunity to hepatitis B (if due to natural infection defined as negative HBsAg, positive hepatitis B antibody [anti-HBs] and positive anti-HBc; if due to vaccination defined as negative HBsAg, negative anti-HVc and positive anti-HBs) are eligible to participate in the study
   i. History or positive test at Screening for hepatitis C virus antibody (anti-HCV)
   j. History or positive test at Screening for human immunodeficiency virus (HIV)
   k. Diagnosed with cancer with metastatic potential within the last 5 years other than carcinoma in situ of the breast or cervix, or basal cell carcinoma of the skin that has been completely excised
   l. Major depressive episode requiring initiation of medication or hospitalization within the previous 90 days
   m. Presence of hallucinations or delusions
   n. Surgery within 12 weeks of screening
   o. For female subjects, pregnancy or ongoing lactation.
5. Any of the following laboratory abnormalities at Screening
   a. Clinically significant (as determined by a cardiologist or local PI) 12-lead ECG abnormalities
   b. Any serum chemistry value (e.g. aspartate aminotransferase [AST], alanine aminotransferase [ALT], alkaline phosphatase, creatine kinase [CK], total bilirubin etc) >2× the upper limit of normal (ULN) on 2 successive determinations less than 2 weeks apart
   c. Serum creatinine above the ULN or estimated glomerular filtration rate (eGFR)<60 mL/min
   d. Platelet count, international normalized ratio (INR), prothrombin time (PT) or partial thromboplastin time (PTT) not within the normal range or other risk for increased or uncontrolled bleeding
   e. Not carrying an APOE 4 allele (e.g. E3/E3; E3/E2; E2/E2).
6. Presence of contraindication to lumbar puncture as judged by local PI.
7. Any other significant medical conditions that, in the opinion of the investigator, would prohibit participation in the study, including inability to tolerate the MRI scan or lumbar puncture procedures.
8. Taking any of the following medications
   a. Antipsychotic agents, including pimavanserin
   b. Stimulant medications
   c. Antidepressant medications whose dose has not been stable for at least 90 days
   d. Immunosuppressant medications, including chronic corticosteroids
   e. Injected or infused antibody therapies, including but not limited antibodies directed against tumor necrosis factor (TNF), anti-interleukin(IL)-6, natalizumab, rituximab and similar agents
   f. Insulin
   g. Anticoagulant or anti-platelet medications including warfarin, heparinoids and direct coagulation factor inhibitors (e.g. apixaban, dagibatran, rivaroxaban)
      either aspirin at a dose of <100 mg/day or clopidogrel at a dose of 75 mg/day, but not both in combination is permitted
9. Participation in any other interventional clinical trial, or treatment with any investigational drug or investigational use of an approved therapy within 30 days (or 5 half-lives of such agent) prior to the first Screening visit.
10. Regular use of cannabis or cannabis products, including non-prescription products containing cannabidiol (CBD)
11. History of drug (including cannabis) or alcohol abuse within the last 5 years

### 5.2.5. Retesting

If laboratory abnormalities during Screening are considered by the Investigator to be transient, then the laboratory tests may be repeated once during Screening. The Investigator's rationale for retesting should be documented. If the retest result is no longer exclusionary, the participant may be enrolled.

### 5.2.6. Rescreening

Participants who have screen-failed are permitted to rescreen once, following consultation with the Medical Monitor. Rescreening may be scheduled after at least 5 days have elapsed from the previous study visit.

### 5.2.7. Withdrawal Criteria

Participation in this clinical study may be discontinued for any of the following reasons:
1. The participant withdraws consent or requests discontinuation from the study for any reason;
2. Occurrence of any medical condition or circumstance that exposes the participant to substantial risk and/or does not allow the participant to adhere to the requirements of the protocol;
3. Any SAE, clinically significant adverse event (AE), severe laboratory abnormality, intercurrent illness, or other medical condition which indicates to the Investigator that continued participation is not in the best interest of the participant;
4. Pregnancy;
5. Requirement of prohibited concomitant medication;
6. Participant failure to comply with protocol requirements or study-related procedures; or
7. Termination of the study by the Sponsor or the regulatory authority.

If a participant withdraws prematurely from the study due to the above criteria or any other reason, study staff should make every effort to complete the full panel of assessments scheduled for the Early Termination Visit. The reason for participant withdrawal must be documented in the electronic case report form (eCRF).

In the case of participants lost to follow-up, at least 3 attempts to contact the participant must be made and documented in the participant's medical records. Withdrawn participants will not be replaced.

### 5.2.8. Study Treatments

Participants are treated with obicetrapib 10 mg per day.

### 5.2.8.1 Rationale for Dosing

In previous multiple-dose clinical studies of obicetrapib in healthy subjects and patients, near maximal effects were observed with the 5 mg obicetrapib dose. At this dose level, CETP activity and concentrations were effectively reduced, and HDL-C levels were increased while LDL-C levels decreased.

### 5.2.8.2 Randomization and Blinding

Participants who meet all eligibility criteria are enrolled into the study. All participants are treated with Obicetrapib 10 mg. Breaking the blind is not applicable, as this an open-label study.

### 5.2.8.3 Drug Supplies

### 5.2.8.3.1 Formulation and Packaging

The study drugs consist of 5 mg obicetrapib tablets. All products are manufactured in accordance with current European Union Good Manufacturing Practice.

Obicetrapib tablets are round, white film-coated tablets, with no identifying markings, containing 5 mg of obicetrapib calcium drug substance. The excipients present in the tablet cores are microcrystalline cellulose, mannitol, sodium starch glycollate, colloidal silicon dioxide, and magnesium stearate. A commercially available film-coating formula (Opadry II white, ex Colorcon) is applied to the cores.

Obicetrapib tablets are packaged into foil blisters and assembled into blister cards. The blister cards are clearly labelled to indicate which blisters to use on each day. Blister cards are assembled into kits, and each kit provides a sufficient supply for the necessary dosing. The shelf-life is assigned based on the stability of the individual products. The kits should be stored below 25° C.

The physical, chemical, and pharmaceutical formulation properties and characteristics of the obicetrapib tablets are described in the Investigator's Brochure.

All study drugs are labelled in accordance with all applicable local regulatory requirements.

### 5.2.8.3.2 Study Drug Preparation and Dispensing

The study drugs used in this study are 5 mg obicetrapib tablets.

At each appropriate visit, participants receive a kit containing blister cards with the study drug appropriate for the participant's treatment group. Participants are instructed to take 2 tablets from the blister cards in the kit each day. The blister cards are clearly labelled to indicate which blisters to use on each day. Each kit provides a sufficient supply for 1 month of dosing. Participants are instructed to return all unused study drugs at the next visit.

### 5.2.8.3.3 Study Drug Administration

Study drugs are administered by the participant orally and once daily on Days 1 through 126. Study drugs should be administered at approximately the same time each morning, with food. On days with visits scheduled, study drugs should be administered with food following all fasted blood samples. If a participant forgets to take study drug on a given day, they should take the next dose as normal and should not take a double dose to make up for the forgotten dose.

### 5.2.8.3.4 Treatment Compliance

Compliance to the study drug regimen is evaluated by counting unused tablets and capsules. During the Treatment Period, if compliance is not between 80% and 120%, inclusive, the participant is counselled about the importance of compliance to the regimen. If the limits are exceeded at 2 consecutive visits, a decision is made by the Investigator and Sponsor as to whether the participant should be withdrawn from the study.

### 5.2.8.3.5 Storage and Accountability

All study drugs are stored below 25° C. in a secure area with access limited to the Investigator and authorized site personnel.

In accordance with regulatory requirements, the Investigator or designated site personnel must document the amount of study drug dispensed and/or administered to participants, the amount returned by participants, and the amount received from and returned to the Sponsor (or representative) when applicable. Study drug accountability records must be maintained throughout the course of the study. Discrepancies are to be reconciled or resolved. Procedures for final disposition of unused study drug are provided in the appropriate study manual.

### 5.2.9. Prior and Concomitant Medications and/or Procedures

5.2.9.1 Excluded Medications and/or Procedures

Any lipid-altering therapy other than the investigational study drug is prohibited during the study.

### 5.2.9.2 Documentation of Prior and Concomitant Medication Use

Medications used within 28 days prior to the Screening Visit are recorded. Any medications administered in addition to the study drugs, whether allowed per the protocol or not, must be documented on the concomitant medication eCRF.

### 5.2.10. Study Procedures

Study procedures follow the Schedule of Procedures:

| Study period | | | | | | |
|---|---|---|---|---|---|---|
| | Treatment Period | | | | | |
| | Baseline | | | | EOT/ET⁶ | Follow- |
| | (V1) | V3 | V4 | V5 | (V5) | Up |
| | Day (±Visit Window) | | | | | |
| Screening | | | D 84 ± | D 126 ± | D 168 ± | |
| 1-8 weeks | D 1 | D 42 | 7 d | 7 d | 7 d | D 198 |

| | | | | | | |
|---|---|---|---|---|---|---|
| Informed consent | X | | | | | |
| Inclusion/exclusion criteria | X | X | | | | |
| Demographic data | X | | | | | |
| Medical history and baseline conditions | X | | | | | |
| Height and weight | X | | | | | X |
| Vital signs | X | | X | X | X | X |
| ECG | X | | | | | X |
| Pregnancy test (if applicable) | X | | | | | |
| Thyroid function | X | | | | | |
| Folic acid and vitamin B12 levels | X | | | | | |
| Hematology, chemistry and urinalysis | X | | | | | X |
| Coagulation | X | | | | | |
| APOE E4 testing | X | | | | | |
| MRI-cerebrum | X | | | | | |
| Physical examination | X | | | X | | X |
| Neurological examination | X | | | X | | X |
| Prior and concomitant medications | X | X | X | X | X | X |
| Dispense study drugs | | X | X | X | X | |
| Study drug administration | | X | X | X | X | X |
| Study drug compliance check | | | X | X | X | X |
| Adverse events | X | X | X | X | X | X |
| CDR-SB | X | X | | | | X |
| MMSE | X | X | | X | | X |
| MOCA | | X | | | | X |
| CFC | | X | | | | X |
| Plasma sample for biomarkers | | X | | X | | X |
| CSF sample for biomarkers | X | | | X⁷ | | X |

APOE E4 = apolipoprotein E-E4; CDR-SB = Clinical Dementia Rating scale-Sum of Boxes; CFC = Cognitive-Functional Composite; ECG = electrocardiogram; EOT = End of Treatment; ET = Early Termination; MMSE = mini-mental state examination; MoCA = Montreal Cognitive Assessment; MRI = magnetic resonance imaging.

### 5.2.10.1.1 Clinical Laboratory Evaluations

Blood for chemistry and hematology are obtained as indicated in the Schedule of Procedures and sent to a central laboratory for analysis. In particular, the following lipid profile data are collected:
Apolipoprotein B
Apolipoprotein E (ApoE)
High-density lipoprotein-ApoE [1]
High-density lipoprotein cholesterol
Low-density lipoprotein cholesterol [2]
Non-high-density lipoprotein cholesterol
Triglycerides
Very low-density lipoprotein cholesterol
Apolipoprotein AI
Apolipoprotein AII
   [note 1] With and without apolipoprotein C3.
   [note 2] Measured by preparative ultracentrifugation, also referred to as beta quantification.

As indicated in the Schedule of Procedures, the following are among the AD biomarker profile data collected in the CSF and/or plasma:
Tau and pTau epitopes

$$A {β}_{1 - 42} , A {β}_{1 - 40} , A {β}_{1 - 42} / A {β}_{1 - 40} ratio$$

Neurogranin
Neurofilament light
GFAP (glial fibrillary acidic protein)
YKL40
PDGFRb.

Blood samples for chemistry and hematology must be obtained under fasting conditions (i.e., after the participant has fasted for ≥10 hours). For the purposes of this study, fasting is defined as nothing by mouth except water and any essential medications. If a participant is not fasting, the Investigator is to reschedule the visit as soon as possible. Estimated glomerular filtration rate is calculated using the Chronic Kidney Disease Epidemiology Collaboration equation. At the Screening Visit only, the hematology panel includes HbA1c, INR, PT and PTT.

Urine is obtained as indicated in the Schedule of Procedures and is sent to a central laboratory for complete urinalysis.

A urine pregnancy test is performed only prior to participation in the study at the screening visit.

### 5.2.10.1.2 Electrocardiograms

A single, standard 12-lead ECG is performed by the Investigator or trained site personnel at the Screening Visit and centrally read.

### 5.2.10.1.3 Physical Examinations

A physical examination is performed as indicated in above Schedule of Procedures.

### 5.2.11. Efficacy Assessments

The primary efficacy endpoint is the change from baseline in levels of ApoA-I, ApoE, and cholesterol efflux capacity in CSF, measured at week 24.

Among secondary efficacy endpoints specific to lipid profile measurement in CSF and plasma, are:
percent change of HDL-C at Screening (Visit 1) or Baseline (Visit 2), at Day 84 (Visit 4), and at the End of Treatment (Day 168, Visit 6).
percent change of HDL subfractions at Screening (Visit 1) or Baseline (Visit 2), at Day 84 (Visit 4), and at the End of Treatment (Day 168, Visit 6).
percent change of HDL-ApoE in HDL particles lacking ApoC3 at Screening (Visit 1) or Baseline (Visit 2), at Day 84 (Visit 4), and at the End of Treatment (Day 168, Visit 6).
percent change of ApoA-II at Screening (Visit 1) or Baseline (Visit 2), at Day 84 (Visit 4), and at the End of Treatment (Day 168, Visit 6).
percent change of 24S-hydroxycholesterol and 27-hydroxycholesterol at Baseline (Visit 2), at Day 84 (Visit 4) and the End of Treatment (Visit 6).

Additional secondary efficacy endpoints specific to AD biomarkers in CSF:
percent change of Tau and pTau epitopes at Screening (Visit 1), at Day 84 (Visit 4), and at the End of Treatment (Day 168, Visit 6).
percent change of Aβ₁₋₄₂, Aβ₁₋₄₀, Aβ₁₋₄₂/Aβ₁₋₄₀ ratio at Screening (Visit 1), at Day 84 (Visit 4), and at the End of Treatment (Day 168, Visit 6).
percent change of neurogranin at Screening (Visit 1), at Day 84 (Visit 4), and at the End of Treatment (Day 168, Visit 6).
percent change of neurofilament light at Screening (Visit 1), at Day 84 (Visit 4), and at the End of Treatment (Day 168, Visit 6).
percent change of GFAP at Screening (Visit 1), at Day 84 (Visit 4), and at the End of Treatment (Day 168, Visit 6).
percent change of YKL40 at Screening (Visit 1), at Day 84 (Visit 4), and at the End of Treatment (Day 168, Visit 6).
percent change of PDGFRb at Screening (Visit 1), at Day 84 (Visit 4), and at the End of Treatment (Day 168, Visit 6).

Additional secondary efficacy endpoints specific to AD biomarkers in plasma are:
percent change of Tau and pTau epitopes at Baseline (Visit 2), at Day 84 (Visit 4), and at the End of Treatment (Day 168, Visit 6).
percent change of Aβ₁₋₄₂, Aβ₁₋₄₀, Aβ₁₋₄₂/Aβ₁₋₄₀ ratio at Baseline (Visit 2), at Day 84 (Visit 4), and at the End of Treatment (Day 168, Visit 6).
percent change of neurogranin at Baseline (Visit 2), at Day 84 (Visit 4), and at the End of Treatment (Day 168, Visit 6).
percent change of neurofilament light at Baseline (Visit 2), at Day 84 (Visit 4), and at the End of Treatment (Day 168, Visit 6).

Additional secondary efficacy endpoints to track disease progression are:
administer the Cognitive-Functional Composite test at Baseline (Visit 2) and End of Treatment (Day 168, Visit 6).
administer the CDR-SB test at Screening (Visit 1) and End of Treatment (Day 168, Visit 6).
administer the Mini-Mental State Examination at Screening (Visit 1), Baseline (Visit 2), Day 84 (Visit 4) and End of Treatment (Day 168, Visit 6).

### 5.2.12. Results

The administration of obicetrapib is effective by End of Treatment to increase plasma levels of total ApoE, HDL-ApoE, HDL-ApoE in particles without Apo-C3, and the percentage of HDL-ApoE in particles lacking ApoC3 in patients who have early AD and at least one ApoE4 allele.

The administration of obicetrapib is effective by End of Treatment to prevent at least one of (i) further decrease in levels of Aβ42 in plasma, (ii) further increase in levels of Aβ40 in plasma, (iii) further decrease in ratio of Aβ42/Aβ40 in plasma, (iv) further increase in levels of neurofilament light (NFL) in plasma, and (v) further increase in levels of neurogranin in plasma, as compared to levels immediately prior to start of treatment.

The administration of obicetrapib is effective to prevent at least one of (i) further decrease in levels of Aβ42 in CSF, (ii) further increase in levels of Aβ40 in CSF, (iii) further decrease in ratio of Aβ42/Aβ40 in CSF, (iv) further increase in levels of neurofilament light (NFL) in CSF, and (v) further increase in levels of neurogranin in CSF, as compared to levels immediately prior to start of treatment.

Subjects experience no significant decline in cognitive status between enrollment and End of Treatment.

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method of slowing progression of neurodegenerative disease in a subject who has or is at risk of developing a neurodegenerative disease, comprising:
administering a therapeutically effective amount of obicetrapib or a pharmaceutically acceptable salt thereof to the subject,
wherein said neurodegenerative disease is Alzheimer's Disease (AD), and wherein the subject has at least one ApoE4 allele.

2. The method of claim 1, wherein the Alzheimer's Disease is AD Clinical Stage 3 or 4 based on the NIA-AA Research Framework criteria.

3. The method of claim 1, wherein the subject does not have mild cognitive impairment (MCI).

4. The method of claim 1, wherein the subject has MCI.

5. The method of claim 1, wherein the subject has been determined, prior to treatment with obicetrapib or a pharmaceutically acceptable salt thereof, to have at least one of
(i) decreased levels of Aβ42 in plasma or CSF,
(ii) increased levels of Aβ40 in plasma or CSF,
(iii) decreased ratio of Aβ42/Aβ40 in plasma or CSF,
(iv) increased levels of neurofilament light (NFL) in plasma or CSF, and
(v) increased levels of neurogranin in plasma or CSF,
as compared to a healthy control population that does not have and that is not at elevated risk for neurodegenerative disease.

6. The method of claim 1, wherein the subject has been determined, prior to treatment, to have abnormal patterns of Tau based on Tau PET imaging.

7. The method of claim 1, wherein the subject is at least 50 years old.

8. The method of claim 7, wherein the subject is at least 60 years old.

9. The method of claim 8, wherein the subject is at least 70 years old.

10. The method of claim 1, wherein the dose of obicetrapib or a pharmaceutically acceptable salt thereof is effective to prevent at least one of:
(i) further decrease in levels of Aβ42 in plasma,
(ii) further increase in levels of Aβ40 in plasma, and
(iii) further decrease in ratio of Aβ42/Aβ40 in plasma,
as compared to levels prior to start of treatment.

11. The method of claim 10, wherein the dose of obicetrapib or a pharmaceutically acceptable salt thereof is effective to prevent further decrease in the ratio of Aβ42/Aβ40 in plasma, as compared to levels prior to start of treatment.

12. The method of claim 11, wherein the dose of obicetrapib or a pharmaceutically acceptable salt thereof is effective to increase the ratio of Aβ42/Aβ40 in plasma, as compared to levels prior to start of treatment.

13. The method of claim 1, wherein obicetrapib or a pharmaceutically acceptable salt thereof is administered orally.

14. The method of claim 13, wherein obicetrapib or a pharmaceutically acceptable salt thereof is administered once per day.

15. The method of claim 14, wherein obicetrapib or a pharmaceutically acceptable salt thereof is administered at a dose of 5-25 mg po QD.

16. The method of claim 15, where the dose is 10 mg po QD of obicetrapib.

17. The method of claim 15, wherein obicetrapib or a pharmaceutically acceptable salt thereof is administered once daily for at least 8 weeks.

18. The method of claim 17, wherein obicetrapib or a pharmaceutically acceptable salt thereof is administered once daily for at least 6 months.

19. The method of claim 13, wherein obicetrapib or a pharmaceutically acceptable salt thereof is administered as a tablet.

20. The method of claim 19, wherein the tablet comprises obicetrapib or the calcium salt thereof.
